(19) United States
(12) Patent Application Publication
(10) Pub. No.: US 2016/0242730 A1
(43) Pub. Date: Aug. 25, 2016

(54) PHYSIOLOGICAL MONITORING DEVICE

(71) Applicant: The Boston Consulting Group, Inc., Boston, MA (US)

(72) Inventors: Christopher Allen RITTENMEYER, Plano, TX (US); Siddharth Kirit SHAH, Duarte, CA (US); Christopher R. WILLIAMS, Manhattan Beach, CA (US); William J. KAISER, Los Angeles, CA (US); Henrik BORGSTROM, Los Angeles, CA (US)

(21) Appl. No.: 15/046,262

(22) Filed: Feb. 17, 2016

Related U.S. Application Data
(60) Provisional application No. 62/119,732, filed on Feb. 23, 2015.

(51) Int. Cl. A61B 7/04; A61B 5/024

(52) U.S. Cl. CPC A61B 7/04; A61B 5/024; A61B 2503/02

(57) ABSTRACT

A physiological monitoring device may comprise a sensor chamber defined by a flexible membrane on a first side and one or more walls and an exterior chamber surrounding the sensor chamber on all sides except for the first side. The sensor chamber may be airtight except for a pressure vent. The exterior chamber may comprise one or more exterior vents and may be arranged to allow for pressure escaping through the pressure vent to equilibrate the sensor chamber when the one or more exterior vents are blocked.

### CROSS REFERENCE TO RELATED APPLICATION

This application claims priority from U.S. Provisional Application No. 62/119,732, filed Feb. 23, 2015, the entirety of which is incorporated by reference herein.

### BRIEF DESCRIPTION OF THE DRAWINGS

FIG. 1 is a cross-sectional view of a physiological monitoring device according to an embodiment of the invention.

FIG. 2 is a perspective view of a sensor chamber according to an embodiment of the invention.

FIG. 3 is a block diagram of a circuit according to an embodiment of the invention.

FIG. 4 is an OPAMP circuit diagram according to an embodiment of the invention.

FIG. 5 is a microcontroller circuit diagram according to an embodiment of the invention.

FIG. 6 is a wireless transmitter circuit diagram according to an embodiment of the invention.

### DETAILED DESCRIPTION OF SEVERAL EMBODIMENTS

Noninvasive, convenient, and low cost systems and methods for acoustic monitoring of fetal and maternal heart beats during pregnancy are described herein. An example monitoring device may use purely passive sensing modalities and, as such, may be completely safe and may be different from various sonar-based fetal monitoring devices.

The example system may include one or several acoustic sensor modules containing microphones whose signals may be amplified and conditioned using an electronic network before being sampled by a microcontroller that may subsequently transmit these raw signals over wireless communication channels to a smartphone, tablet device, or other computer which may include special-purpose hardware, firmware, and/or software for subsequent data analysis and algorithms.

The sensor modules may contain one or more microphones (e.g., an electret or MEMS microphone) and/or other sensors housed within an enclosure that may be optimized for mechanical amplification of cardiac or abdominal acoustic emissions. During use, this module may be held against the abdomen or upper pubis of the subject. The enclosure may be cylindrical in shape or may assume the shape of a parabolic, elliptical, or cone-shaped acoustic amplifier horn, for example. The surface of the sensor module that is configured to contact the abdomen may be sealed (e.g., with a polymer, rubber, or latex material) to create an airtight chamber within which the microphone is housed. In addition to the microphone, the sensor module may house one or more circuits. For example, the sensor module may house a printed circuit board (PCB) which may include an operational amplifier (OPAMP) or other analog signal conditioning network, a microcontroller unit, a USB or other charging and/or data port, a Bluetooth radio and/or other wireless device, a battery or other power supply, and/or other hardware. The hardware may provide a built-in analog band pass filter to enhance dynamic range, provide desirable performance, and limit requirements for external data acquisition and/or transmission systems.

Signal processing hardware, firmware, and/or software may be hosted on a remote device such as a tablet or smartphone running either and Android or iOS operating system, for example. These elements may use algorithms to improve the signal to noise ratio (SNR) of the captured signal, compute maternal and fetal heart rates, isolate maternal and fetal heart sounds, reconstruct acoustic signals of the mother and fetus, and/or provide high quality audio files for recording, playback, and/or sharing via a software application. The signal processing may discriminate and extract fetal heart sound from other sounds such as ambient noise, maternal heartbeat sound, digestive motility sound, peristaltic sound, and/or other sounds. The signal may be extracted even with uncertain sensor coupling, sensor location, and/or signal characteristics.

The physical monitoring device may be a hand-held apparatus including one or several sensor modules containing electret or MEMS microphones, a signal conditioning network that performs amplification and anti-aliasing, a microcontroller device that samples the conditioned signal, and/or a Bluetooth radio module that transmits acquired data to a backend smartphone or tablet device. Other components may include a Lithium-ion charger with rubber protective seal, Lithium polymer battery, rubber seal for water resistance, a plastic baseboard to prevent penetration of the neoprene seal, and other electronic components to complete the printed circuit board assembly. Those of ordinary skill in the art will appreciate that other components may be used in other embodiments (e.g., other sensor types, other controllers, other wireless or wired transmitters, other power supplies, other module components, etc.).

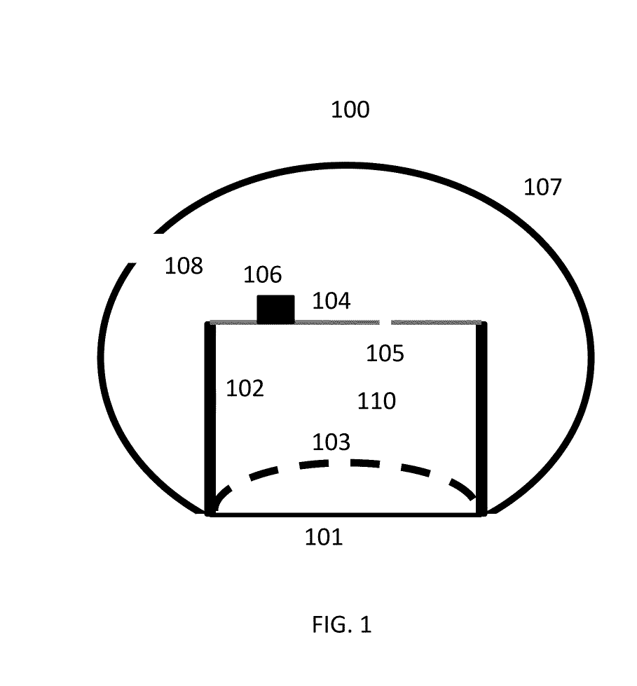

Each microphone may be contained in an airtight enclosure optimized for mechanical amplification of acoustic and pressure signatures associated with fetal and maternal heart activity. For example, FIG. 1 is a cross-sectional view of a physiological monitoring device 100 according to an embodiment of the invention. The device 100 may include a nearly airtight sensor chamber 110 defined by a membrane 101, walls 102, and an electronic printed circuit board (PCB) 104. The membrane 101 may be made of elastomer material in some embodiments, and the walls 102 may be plastic in some embodiments, but other materials may be used. PCB 104 may be impermeable to air except for a small hole 105 (which may be approximately 0.6 mm in diameter in some embodiments, for example) which may allow for venting of pressure from the chamber 110. The sensor chamber 110 may be entirely airtight except for this feature. The hole 105 may allow for venting of low-frequency pressure changes due to modulation of application pressure or other disturbances. This may reduce the gain of the system at low frequencies that may not contain fetal heartbeat sounds without affecting the sensitivity of the system at higher frequencies. Accordingly, the sensor chamber 110 may serve as a mechanical high pass filter removing inputs with frequencies lower than approximately 20 Hz, for example.

A microphone 106 may be mounted on the top of the PCB 104 aiming downward towards the sensor chamber 110, and a hole in the PCB 104 covered by the microphone 106 may allow acoustic energy to pass through to the microphone 106. The interface between the microphone 106 and the PCB 104 may be formed by a two-sided adhesive and may be airtight. Similarly, the PCB 104 may be attached to the walls 102 in an airtight fashion using a two-sided adhesive. Other adhesives, such as epoxy or cyanoacrylate, may be used in some embodiments.

A protective grid 103 may be provided to protect the membrane 101 from excessive deflection as well as to prevent the user from contacting any electronic elements as a safety feature. The grid 103 may be made from the same material as the walls 102 in some embodiments (e.g., ABS or other plastic). The grid 103 may be curved inward in some embodiments as shown in FIG. 1. This may allow the membrane 101 to deform inward when pressed against a user's skin to prevent or reduce user discomfort.

The sensor chamber 110 may be mounted inside an exterior chamber 107. This chamber 107 may include one or more vents 108 and is thereby not airtight, allowing for pressure vented from the sensor chamber 110 to escape the device 100 and thereby equilibrate quickly. However, even if a user accidentally covers these vents, the larger volume of the exterior chamber 107 relative to the sensor chamber 110 may allow for effective venting of pressure from the sensor chamber 110 until the vents 108 are uncovered by the user.

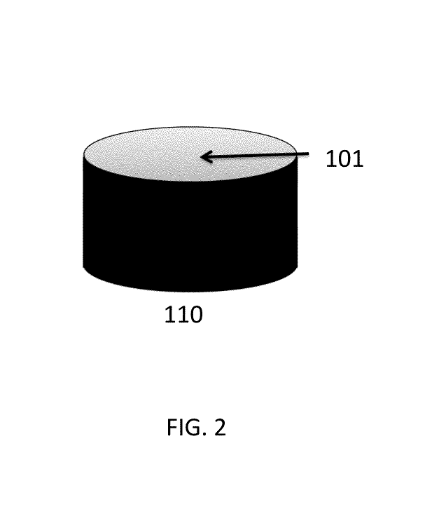

FIG. 2 is a perspective view of a sensor chamber 110 according to an embodiment of the invention. For example, the sensor chamber 110 may be a plastic cylindrical tube with internal diameter of approximately 32 mm and height of approximately 22 mm, although other enclosures having different internal volumes may be used in some embodiments. The shape of the enclosure may be chosen to minimize ambient noise registered by the microphone while maximizing amplification of the microphone.

The membrane 101 may be made of a latex or other elastomer material such as neoprene with an elastomer coating, for example. Other example materials may include santoprene or silicone. Neoprene, santoprene, or silicone may provide a flexible enclosure, and the elastomer coating may strengthen the neoprene, santoprene, or silicone and provide a shiny surface for the enclosure. The latex or other elastomer material may be designed to provide improved impedance matching with human tissue and may be flexible enough to conform to the contours of the user's skin, thereby enhancing transfer of acoustic signals into the sensor chamber 110.

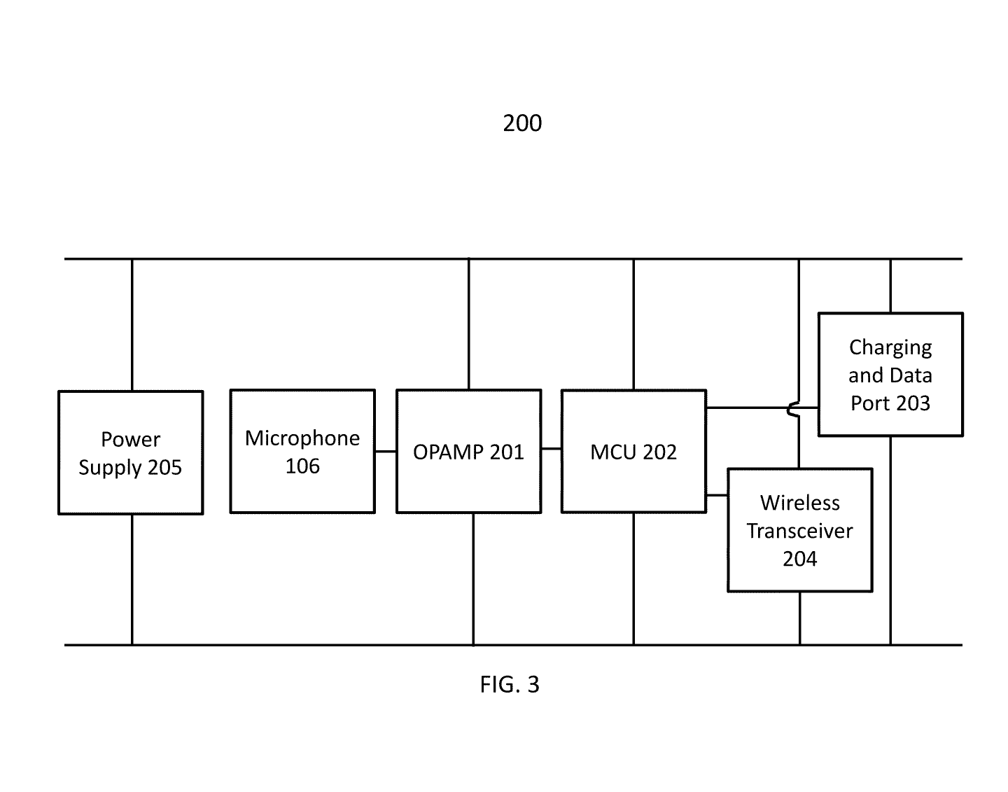

FIG. 3 is a block diagram of a circuit 200 according to an embodiment of the invention. The circuit 200 may be formed entirely, or in part, on the PCB 104. The circuit 200 may include the microphone 106, an OPAMP 210, a microcontroller unit 202, a USB or other charging and/or data port 203, a Bluetooth radio and/or other wireless transmitter or transceiver 204, a battery or other power supply 205, and/or other hardware. The circuit 200 may perform processing associated with capturing signals from the microphone 106 and sending data to a remote device (e.g., via the data port 203 and/or wireless transmitter 204).

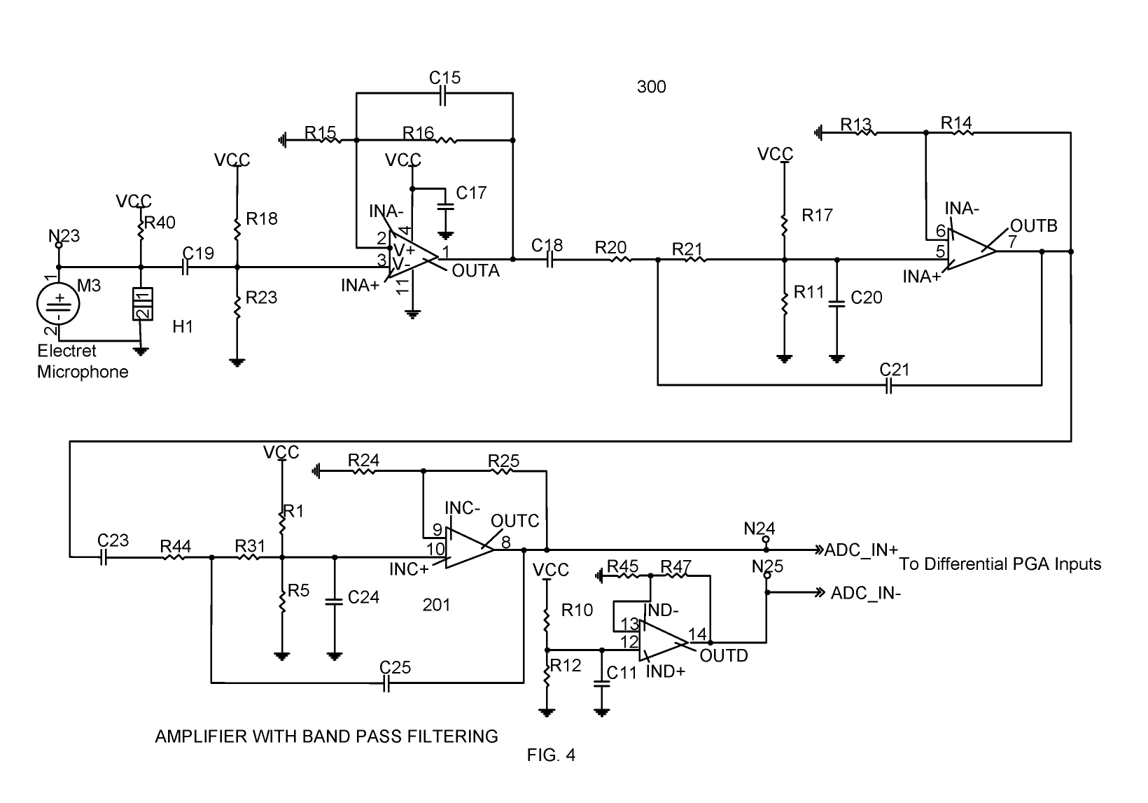

For example, because the acoustic emissions associated with fetal heart events are characterized by very low amplitude, signals captured by the microphone 106 may be amplified and/or conditioned. To this end, the OPAMP 210 may be used to provide gain and anti-aliasing capabilities. To avoid saturation, a relatively low gain (e.g., 20 dB) may be used. The OPAMP 210 used may be chosen to optimize other amplifier parameters such as high-pass and anti-alias filters. FIG. 4 is an OPAMP circuit 300 diagram according to an embodiment of the invention, including the OPAMP 210 and related circuit elements. The OPAMP circuit 300 may include a multi-stage analog amplifier with band pass filtering. The OPAMP circuit 300 may reach an overall gain of about 20 dB at around 40 Hz, resulting in the amplification of input heart beat signal. The frequency response may be such that the signal rolls off on the low end (˜15 Hz), followed by a sharp increase in gain peaking at about 40 Hz. As the frequency of input signal increases, the OPAMP circuit 300 may filter off higher frequencies to dampen signals to about 40 dB below the peak gain value, producing a narrow frequency response that may condition fetal heart beat signals and reject other frequencies/noises.

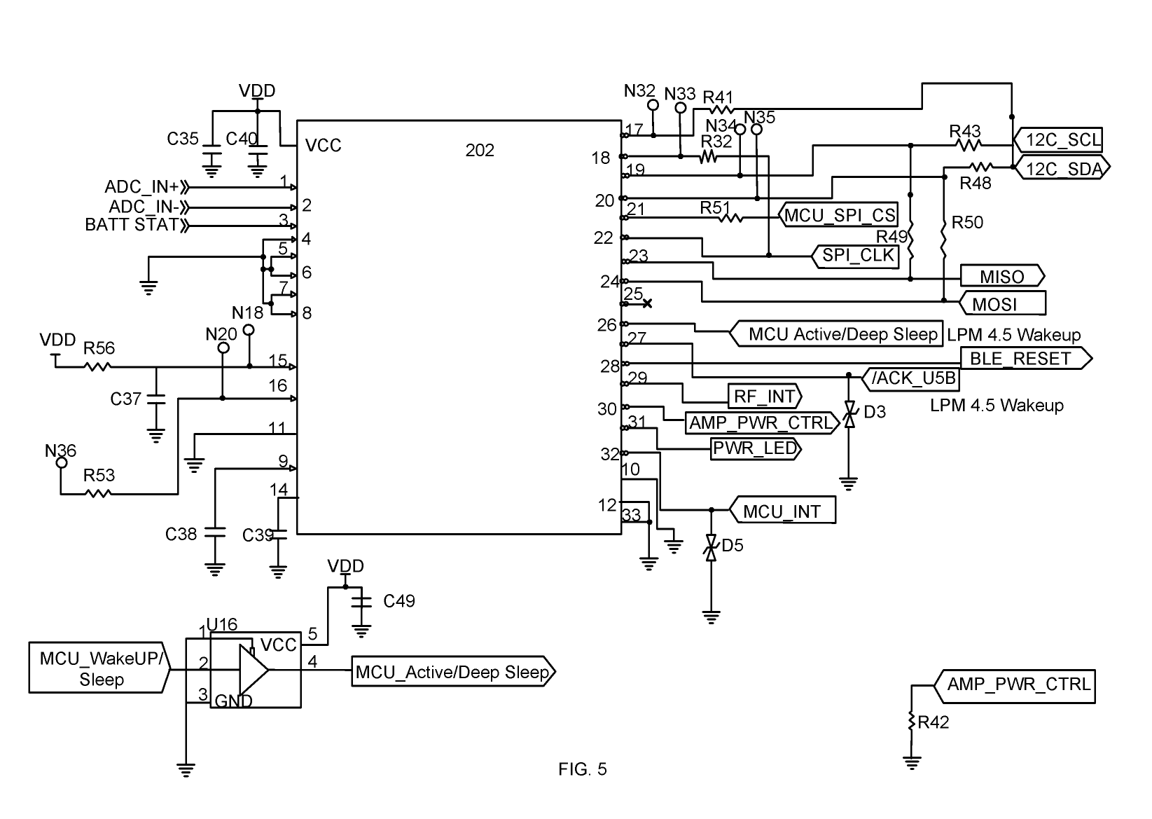

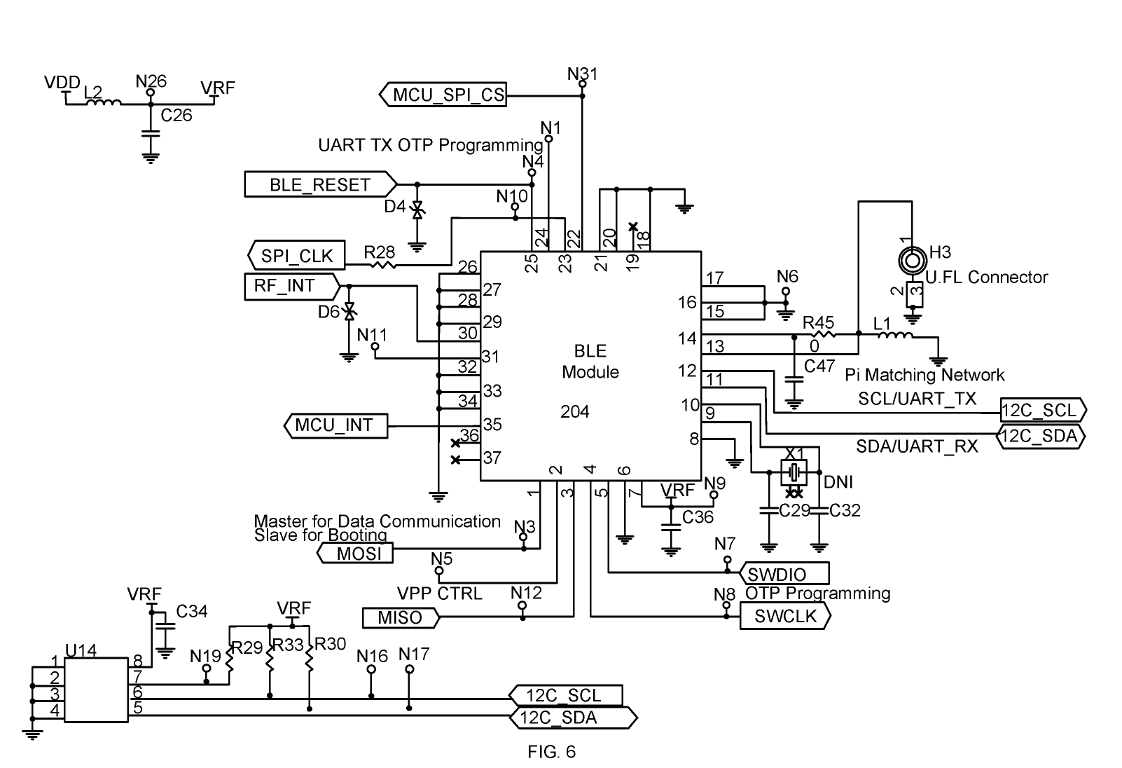

The amplified and conditioned analog signal may be sampled by the microcontroller 202 through the microcontroller's onboard analog to digital converter (ADC) capabilities. For example, the microcontroller 202 may be an MSP43012021 from Texas Instruments or a RFDUINO (nRF51822) from Nordic Semiconductor. In some cases, the microcontroller 202 may include a wireless transmitter 204. For example, the nRF51822 features a 32-bit ARM Cortex M0 core integrated with a Bluetooth Smart® radio device (i.e., wireless transmitter 204). In other cases (e.g., when the MSP43012021 is used), the wireless transmitter 204 may be a standalone element, such as an LBCA2HNZYZ certified BLE radio module from Murata Electronics. FIG. 5 is a microcontroller circuit 202 diagram according to an embodiment of the invention, illustrating how an MSP43012021 may be configured to function within the circuit 200. FIG. 6 is a wireless transmitter 204 circuit diagram according to an embodiment of the invention, illustrating how an LBCA2HNZYZ may be configured to function within the circuit 200.

The microcontroller 202 may sample the amplified microphone signal (e.g., at a rate of 1-2 kHz) and temporarily store data in buffers before transmitting it at time intervals (e.g., roughly 50 mS) via the wireless transmitter 204. This relatively low sample rate may be capable of accurately capturing heart sounds, which are characterized by low frequencies typically below 200 Hz. Other microcontrollers 202 may be used in other embodiments and may perform similar functions.

The microcontroller 202 may perform housekeeping tasks, such as continuously monitoring or periodically checking inputs and performing appropriate operations in response to user inputs. The microcontroller 202 may be able to detect when a USB is plugged in for battery recharging purposes. The microcontroller 202 may also measure system battery health and communicate battery health data to a remote device via the wireless transmitter 204, for example. Via the separate OPAMP analog measurement circuit 300 and a built-in ADC, the microcontroller 202 may determine the system battery status and communicate the system battery status to a remote device via wireless transmitter 204. Other power management circuitry may include LDO voltage regulators to regulate system power and an analog comparator/PFET circuit that may cut off the main power to the system when a low battery is detected. A push button controller circuit may enable system activation and shutdown when a button press is detected for at least a pre-determined amount of time. A suitable battery charger IC may be used to charge the on board battery at a pre-determined rate upon plugging the device into a USB power source.

The wireless transmitter 204 may transmit this data to a remote processing device such as a such as a tablet or smartphone running an Android, iOS or other operating system. Known or proprietary signal processing algorithms may be hosted on the remote device. The algorithms may improve the signal to noise ratio (SNR) of the captured signal, compute maternal and fetal heart rates, isolate maternal and fetal heart sounds, reconstruct acoustic signals of the mother and fetus, and/or provide high quality audio files for recording, playback, and sharing via the software application.

While various embodiments have been described above, it should be understood that they have been presented by way of example and not limitation. It will be apparent to persons skilled in the relevant art(s) that various changes in form and detail can be made therein without departing from the spirit and scope. In fact, after reading the above description, it will be apparent to one skilled in the relevant art(s) how to implement alternative embodiments.

In addition, it should be understood that any figures that highlight the functionality and advantages are presented for example purposes only. The disclosed methodologies and systems are each sufficiently flexible and configurable such that they may be utilized in ways other than that shown.

Although the term “at least one” may often be used in the specification, claims and drawings, the terms “a”, “an”, “the”, “said”, etc. also signify “at least one” or “the at least one” in the specification, claims, and drawings.

Finally, it is the applicant's intent that only claims that include the express language “means for” or “step for” be interpreted under 35 U.S.C. 112(f). Claims that do not expressly include the phrase “means for” or “step for” are not to be interpreted under 35 U.S.C. 112(f).

1. A physiological monitoring device comprising:
a sensor chamber defined by a membrane on a first side and one or more walls, the sensor chamber being airtight except for a pressure vent; and
an exterior chamber substantially surrounding the sensor chamber on all sides except for the first side and comprising one or more exterior vents, the exterior chamber being arranged to allow for pressure escaping through the pressure vent to equilibrate the sensor chamber when the one or more exterior vents are blocked.

2. The device of claim 1, wherein the sensor chamber is further defined by a printed circuit board (PCB) on a second side opposite the first side.

3. The device of claim 2, wherein the one or more walls comprise a substantially cylindrical wall separating the membrane and the PCB.

4. The device of claim 2, wherein the PCB houses a circuit comprising a microphone arranged to detect sound within the sensor chamber.

5. The device of claim 4, wherein the circuit further comprises:
an amplifier configured to amplify a microphone output;
a processor configured to process an amplified output from the amplifier; and
a transmitter configured to transmit a processed output from the processor.

6. The device of claim 4, wherein the circuit further comprises an analog band pass filter configured to filter a microphone output.

7. The device of claim 1, further comprising a microphone arranged to detect sound within the sensor chamber.

8. The device of claim 7, further comprising a circuit comprising:
the microphone;
an amplifier configured to amplify a microphone output;
a processor configured to process an amplified output from the amplifier; and
a transmitter configured to transmit a processed output from the processor.

9. The device of claim 1, further comprising a grid disposed within the sensor chamber and arranged to restrict deflection of the membrane.

10. The device of claim 1, wherein the sensor chamber is configured as a mechanical high pass filter.

11. The device of claim 1, wherein the mechanical high pass filter removes inputs with frequencies lower than approximately 20 Hz.

12. The device of claim 1, wherein the sensor chamber is configured as a cylindrical tube with an internal diameter of approximately 32 mm and a height of approximately 22 mm.

13. The device of claim 1, wherein the pressure vent is approximately 0.6 mm in diameter.

14. The device of claim 1, wherein the exterior chamber defines a volume between an outside of the sensor chamber and an inside of the exterior chamber.

15. The device of claim 14, wherein the pressure escaping through the pressure vent is vented into the volume defined by the exterior chamber.

16. The device of claim 13, wherein the pressure escaping through the pressure vent is vented into the volume defined by the exterior chamber.

17. The device of claim 1, wherein the membrane comprises an elastomeric material.

18. The device of claim 17, wherein the elastomeric material comprises latex, neoprene, silicone rubber, santoprene, elastomeric coated neoprene, or any combination thereof.

19. The device of claim 1, wherein the exterior chamber comprises a vent.
